(19) United States
(12) Patent Application Publication
(10) Pub. No.: US 2026/0100269 A1
(43) Pub. Date: Apr. 9, 2026

(54) METHODS AND SYSTEMS OF FACILITATING PHARMACY MANAGEMENT

(71) Applicant: Omar Hassad, Mokena, IL (US)

(72) Inventor: Omar Hassad, Mokena, IL (US)

(21) Appl. No.: 18/906,569

(22) Filed: Oct. 4, 2024

(51) Int. Cl. G16H 40/20; G06Q 10/087; G16H 20/13

(52) U.S. Cl. CPC G16H 40/20; G06Q 10/087; G16H 20/13

(57) ABSTRACT

The present disclosure provides a method of facilitating pharmacy management. Further, the method may include receiving two or more medicine data from one or more of a medicine database and a platform device. Further, each of the two or more medicine data corresponds to a unique medicine code. Further, the method may include receiving a medicine search code and a transaction type data from a user device associated with a user. Further, the method may include comparing the medicine search code and the unique medicine code. Further, the method may include identifying an identified medicine data from the two or more medicine data based on the comparing. Further, the method may include updating an inventory data stored in a storage device based on the identified medicine data and the transaction type data.

### FIELD OF DISCLOSURE

Generally, the present disclosure relates to the field of data processing. More specifically, the present disclosure relates to methods and systems for facilitating pharmacy management.

### BACKGROUND

Pharmacies provide necessary medicated drugs to many patients and other pharmacies with deficient in particular drugs. Operating these pharmacies often includes managing the stocks of available drugs and keeping track of all the transactions that happened over a while. In existing technologies keeping track of all these transactions is time-consuming and tedious. Current technologies often do not include a feature to integrate a user dealing with multiple wholesalers and other pharmacies alike in a single platform, therefore regularly requiring multiple software platforms to keep track of all the records. In current technologies, the user may use manual techniques to keep track of all the records.

Therefore, there is a need for systems and methods of facilitating provisioning of an aid corresponding to a grievance of a user that may overcome one or more of the above-mentioned problems and/or limitations.

### SUMMARY OF DISCLOSURE

This summary is provided to introduce a selection of concepts in a simplified form that are further described below in the Detailed Description. This summary is not intended to identify key features or essential features of the claimed subject matter. Nor is this summary intended to be used to limit the claimed subject matter's scope.

The present disclosure provides a method of facilitating pharmacy management. Further, the method may include receiving, using a communication device, two or more medicine data from one or more of a medicine database and a platform device. Further, each of the two or more medicine data corresponds to a unique medicine code. Further, the method may include receiving, using the communication device, a medicine search code and a transaction type data from a user device associated with a user. Further, the method may include comparing, using a processing device, the medicine search code, and the unique medicine code. Further, the method may include identifying, using the processing device, an identified medicine data from the two or more medicine data based on the comparing. Further, the method may include updating, using the processing device, an inventory data stored in a storage device based on the identified medicine data and the transaction type data.

The present disclosure provides the system for facilitating pharmacy management. Further, the system may include a communication device. Further, the communication device may be configured to receive two or more medicine data from one or more of a medicine database and a platform device. Further, each of the two or more medicine data corresponds to a unique medicine code. Further, the communication device may be configured to receive a medicine search code and a transaction type data from a user device associated with a user. Further, the system may include a processing device communicatively coupled with the communication device. Further, the processing device may be configured to compare the medicine search code and the unique medicine code. Further, the processing device may be configured to identify an identified medicine data from the two or more medicine data based on the comparing. Further, the processing device may be configured to update, an inventory data stored in a storage device based on the identified medicine data and the transaction type data.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 illustrates a flowchart of a method 300 of facilitating pharmacy management, in accordance with some embodiments.

FIG. 4 illustrates a flowchart of a method 400 of facilitating pharmacy management including determining, using the processing device 504, a description data associated with the medicine search code, in accordance with some embodiments.

FIG. 5 illustrates a block diagram of a system 500 of facilitating pharmacy management, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and is made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.” The following detailed description refers to the accompanying drawings.

Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice-based interface, gesture based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal, or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer, etc.) and/or possession of a machine readable secret data (e.g. encryption key, decryption key, bar codes, etc.) and/or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performances of any two steps of the one or more steps.

Further, in some embodiments, the one or more predefined conditions may be specified by the one or more users. Accordingly, the one or more steps may include receiving, using the communication device, the one or more predefined conditions from one or more devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data, and any intermediate data there between corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

C2e log is a compliance feature that makes it easy for a user to keep records of inventory of the user's pharmacies. It is essential that the user have at least one pharmacist on staff list on the C2e log platform. The User can add staff under the compliance tab on the staff page.

The first step to start using the C2e log is to add the current inventory of the user. The current inventory can be added by hovering a cursor over the C2e log tab and selecting Distributors then clicking on add new facility. The user then enters Pharmacy name address and phone number of the user.

After adding all the inventory come back to the C2e log page to add distributors and reverse vendors. It is recommended to label the reverse vendors to facilitate ease of identification. The user can always add more facilities if required. To log the current C2 inventory go to the e-log tab then click on the receive button and type in at least the first nine digits of an NDC. The user will see options automatically appear in a drop-down, select the desired C2 medication and all the required fields such as product name, strength, and pack size will autofill. It is important that the user always searches by NDC and not by product name or any other field, further do not edit any of the product details, this ensures DEA compliance and the most accurate records. The user is entering current C2 inventory so use the invoice number field to note it. Further, enter the total quantity the user has of the entered NDC for example if the user has five packs of 100 tablets, the user should enter “500” and then select the Pharmacy of the user as the distributor. Finally, select the pharmacist documenting the inventory and click submit. Repeat these steps for each NDC the user currently has in inventory.

When the inventory is logged, the user may start tracking received inventory from distributors of the user as well as dispensed lost or damaged expired and returned medications. Note that the user must log a medication as received before the user may record any of the other actions to log a received C2 from one of the distributors of the user, click on the receive button, and start typing the NDC. Always search by NDC and select from the drop-down, the user should never have to fill out the product details manually anywhere on the platform. Then fill out the activity details and click submit.

Since the platform already had a record of the entered NDC, the quantity was added to a quantity on hand.

To track a dispensed C2 click on the dispense button and type the first four digits of the NDC for medications from the log to appear in the drop-down. Select the correct option then fill out the activity details. Once the user clicks submit, the user may see the dispensed activity has been logged and the quantity on hand has decreased by the number of medications dispensed.

To track a lost or damaged C2, click on the Lost/Damage button, type at least the first four digits of the NDC, and select an option from the drop-down. In the activity details the user must fill out the quantity, documenting pharmacist and a node explaining what happened to the medication. Click submit and the user may see the quantity on hand has decreased by the number that was lost or damaged. The user can view the note by hovering over the green knot.

To track an expired C2 click on the expired button, start typing the NDC select an option from the drop down enter the quantity, and documenting pharmacist. Then select an expired type, the option includes a quarantine option meaning that the product is expired but the user is not sending it to the reverse vendor immediately. Select the date of record and click submit. The user may see the expired quarantine activity has been recorded and the expired quantity has been added to the quarantine count the quantity on hand has also decreased by the amount quarantine. To record sending quarantined or expired medications to the reverse vendor, click on the expired button and enter the required information including the total quantity of expired, and quarantined medication the user is sending to the reverse vendor. Select reverse vendor under expired type, the user may have to select the reverse vendor from the distributor drop-down. Once the user clicks submit, the user may notice that the quantity entered is first subtracted from the quarantine count and then from the quantity on hand.

To track medications that need to be returned to the distributor, click on the return button, start typing the NDC select the correct medication then fill out the activity details. In the notes type the reason for the return. Once the user clicks submit the user may be able to see this activity and view the note by hovering over the green knot.

The last feature on the e-log page is audit and event. Start typing the NDC the user is auditing and select the correct medication. Enter the total quantity of the medication at the Pharmacy of the user including on hand and in quarantine. If this total does not match the total from the log, the user may be required to provide a reason and the quantity on hand will be amended automatically.

C2e log lets the user see all activity for a specific medication in one place. To find any drug in the log, type in at least the first five characters of the NDC or drug name. The activity column will always show the last recorded activity. To see a detailed log of every activity related to that NDC, click the plus button.

When the user needs to produce a report, click on the download report option. The user may choose to download an on-hand report which will give the user a quick overview of all the current inventory including quarantined and on-hand quantities. The detailed report option allows the user to choose start and end dates that are at least a month apart. The report shows every activity the user has logged in the chosen time frame grouped by NDC. For either report, the user may filter by a specific product name or NDC by typing in at least the first five characters of the NDC or drug name.

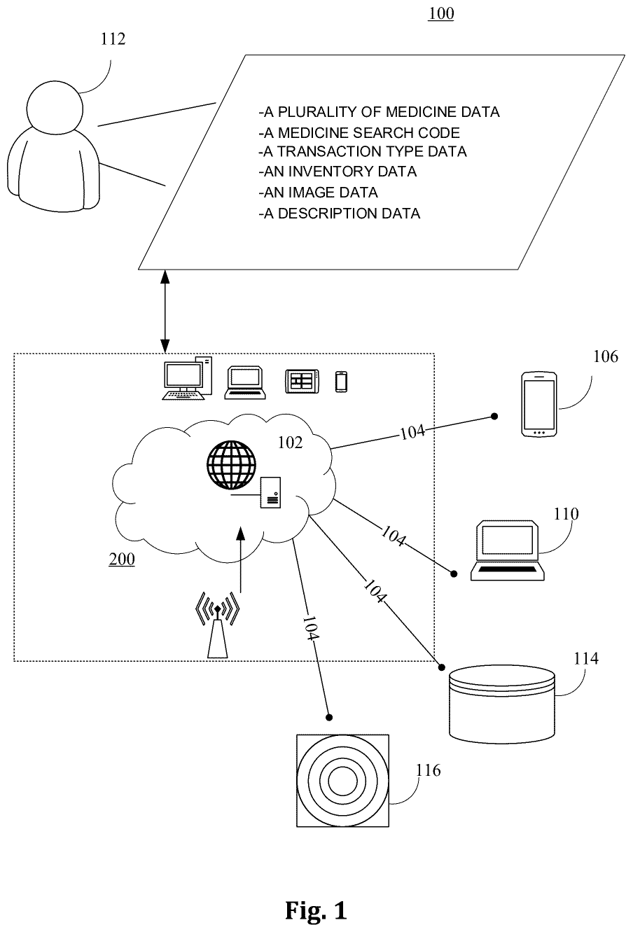

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 200.

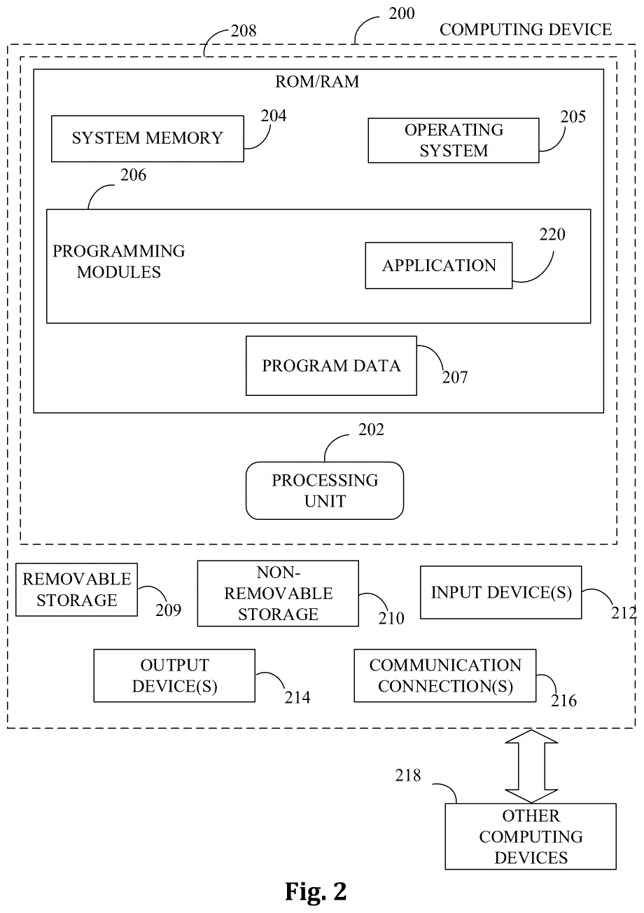

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g. random-access memory (RAM)), non-volatile (e.g. read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable, and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include but is not limited to, RAM, ROM, electrically erasable read-only memory (EEPROM), flash memory or other memory technology, CD-ROM, digital versatile disks (DVD), or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods'stages may be modified in any manner, including by reordering stages and/or inserting or deleting stages, without departing from the disclosure.

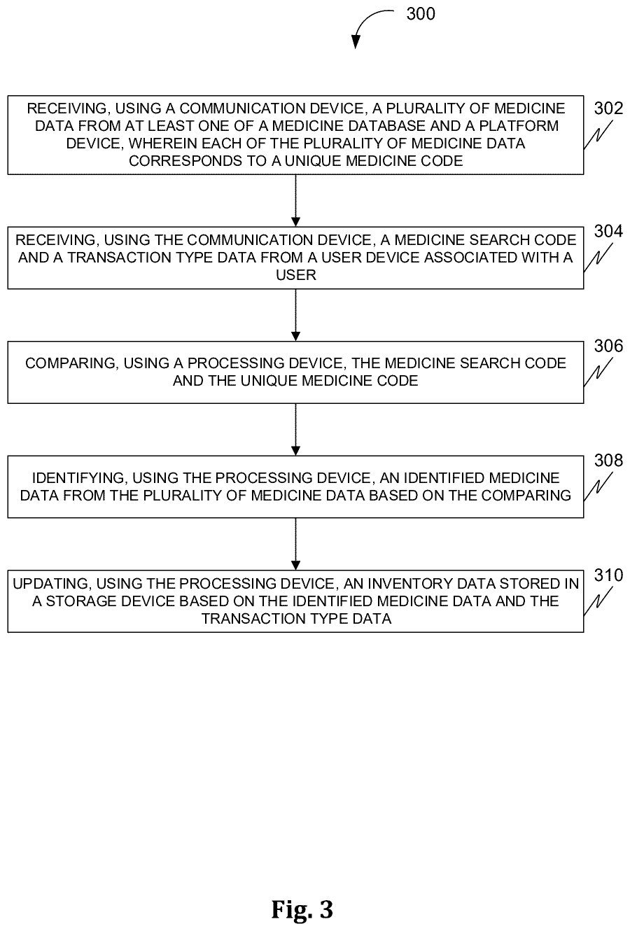

FIG. 3 illustrates a flowchart of a method 300 of facilitating pharmacy management, in accordance with some embodiments.

In some embodiments, the pharmacy includes a store that may be configured to facilitate one or more of a sale, a purchase, and a stockpiling of a medicine. In some embodiments, the facilitation of one or more of the sales, the purchase may be based on a prescription associated with a patient. Further, the prescription may be generated by a healthcare professional.

In some embodiments, the pharmacy management includes an inventory management associated with the pharmacy.

Accordingly, the method 300 may include a step 302 of receiving, using a communication device 502, two or more medicine data from one or more of a medicine database and a platform device.

In some embodiments, the communication device 502 includes an electronic device which may be configured to facilitate one or more of a receiving and a transmitting of a data. In some embodiments, one or more of the receiving and the transmitting may be wireless.

In some embodiments, the medicine database is associated with a legislative jurisdiction.

In some embodiments, each of the two or more medicine data includes one or more of a medicine name, a medicine composition data, a medicine dosage data, a medicine manufacturer data, and a medicine packaging data.

In some embodiments, the platform device includes one or more of a personal computer, a smart device, and a server.

Further, each of the two or more medicine data corresponds to a unique medicine code.

In some embodiments, the unique medicine code includes a National Drug Code.

Further, the method 300 may include a step 304 of receiving, using the communication device 502, a medicine search code, and a transaction type data from a user device associated with a user.

In some embodiments, the medicine search code includes a National Drug Code.

In some embodiments, the user device includes one or more of a smart device and a personal computer.

In some embodiments, the user includes a pharmacist.

Further, the method 300 may include a step 306 of comparing, using a processing device 504, the medicine search code, and the unique medicine code.

In some embodiments, the processing device 504 includes an electronic device which may be configured to execute a set of instruction.

Further, the method 300 may include a step 308 of identifying, using the processing device 504, an identified medicine data from the two or more medicine data based on the comparing.

In some embodiments, the identification of the identified medicine data may be based on an identical match of the unique medicine code and the medicine search code.

Further, the method 300 may include a step 310 of updating, using the processing device 504, an inventory data stored in a storage device based on the identified medicine data and the transaction type data.

In some embodiments, the inventory data includes one or more of the identified medicine data, the medicine search code, the transaction type data, a quantity associated a medicine corresponding to the medicine search code, a transaction date, a documenter name, a distributor data, and an invoice id data.

In some embodiments, the storage device includes a non-volatile memory. In some embodiments, the storage device may be associated with a cloud computing service.

In some embodiments, the method 300 may further include receiving, using the communication device 502, the inventory data from the user device.

In some embodiments, the method 300 may further include transmitting, using a communication device 502, a user interface to the user device comprising a user processing device, a user communication device, and a user presentation device. Further, the user presentation device may be configured to present the user interface to the user.

Further, the user interface may be configured to receive a user input associated with the transaction type data. Further, the receiving of the transaction type data may be based on the user input.

In some embodiments, the receiving of the transaction type data may be based on an external sensor data generated by an external sensor associated with a medicine corresponding to the medicine search code.

In some embodiments, the external sensor includes an RFID sensor which may be configured to detect signal from an RFID tag associated with a medicine associated with the medicine search code.

In some embodiments, the method 300 may further include receiving, using the communication device 502, a description data associated with the medicine search code. Further, the updating of the inventory data may be based on the description data.

In some embodiments, the description data includes one or more of a quantity of a medicine associated with the medicine search code, a documenter data, an invoice data, a date data, and a facility data.

In some embodiments, the inventory data includes one or more of the identified medicine data, the medicine search code, the transaction type data, a quantity associated a medicine corresponding to the medicine search code, a transaction date, a documenter name, a distributor data, and an invoice id data.

In some embodiments, the transaction type data includes indication of a receiving of a medicine corresponding to the medicine search code. Further, the updating of the inventory data may be based on the receiving of the medicine.

In some embodiments, the method 300 may further include receiving, using the communication device 502, a received quantity data of the medicine. Further, the inventory data includes a stock quantity data associated with the medicine search code.

Further, the updating of the inventory data includes updating the stock quantity data may be based on the received quantity data.

In some embodiments, the transaction type data includes indication of a dispensing of a medicine corresponding to the medicine search code. Further, the updating of the inventory data may be based on the dispensing of the medicine.

In some embodiments, the method 300 may further include receiving, using the communication device 502, a dispensed quantity data of the medicine. Further, the inventory data includes a stock quantity data associated with the medicine search code. Further, the updating of the inventory data includes updating the stock quantity data may be based on the dispensed quantity data.

In some embodiments, the transaction type data includes indication of one or more of a loss and a damage of a medicine corresponding to the medicine search code. Further, the updating of the inventory data may be based on a quantity associated with one or more of the loss and the damage of the medicine.

In some embodiments, the method 300 may further include receiving, using the communication device 502, a loss quantity data of the medicine. Further, the inventory data includes a stock quantity data associated with the medicine search code. Further, the updating of the inventory data includes updating the stock quantity data may be based on the loss quantity data.

In some embodiments, the transaction type data includes indication of an expiration of a medicine corresponding to the medicine search code. Further, the updating of the inventory data may be based on the expiration of the medicine.

In some embodiments, the method 300 may further include receiving, using the communication device 502, an expired medicine quantity data of the medicine. Further, the inventory data includes a stock quantity data associated with the medicine search code. Further, the updating of the inventory data includes updating the stock quantity data based on the expired medicine quantity data.

In some embodiments, the transaction type data includes indication of an audit of the inventory data associated with a medicine associated with the medicine search code.

Further, in some embodiments, the method 300 further may include a step of receiving, using the communication device 502, a present stock data of the medicine. Further, in some embodiments, the method 300 further may include a step of comparing, using the processing device 504, the present stock data and a stock data associated with the medicine search code. Further, the stock data may be associated with the inventory data. Further, in some embodiments, the method 300 further may include a step of receiving, using the communication device 502, a reason data associated with the medicine based on the comparing.

In some embodiments, the transaction type data includes indication of an amending of the inventory data associated with a medicine associated with the medicine search code.

Further, in some embodiments, the method 300 further may include a step of receiving, using the communication device 502, a present stock data of the medicine. Further, in some embodiments, the method 300 further may include a step of updating, using the processing device 504, the inventory data associated with the medicine based on a present stock data.

In some embodiments, the transaction type data includes indication of a return of a medicine corresponding to the medicine search code. Further, the updating of the inventory data may be based on the return of the medicine.

In some embodiments, the method 300 may further include receiving, using the communication device 502, a return medicine quantity data of the medicine. Further, the inventory data includes a stock quantity data associated with the medicine search code. Further, the updating of the inventory data includes updating the stock quantity data may be based on the return medicine quantity data.

In some embodiments, the method 300 may further include generating, using the processing device 504, a digital report data associated with the inventory data.

In some embodiments, the storage device may be a node of a blockchain network.

In some embodiments, the blockchain network includes a decentralized storage system. Further, a data may be stored in two or more blocks in the decentralized storage system.

In some embodiments, the updating of the inventory data may be caused by generation of a block data based on the transaction type data and the medicine search code. In some embodiments, each of a block in the two or more blocks may be configured to connect with a previous block in the two or more blocks based on a cryptographic function.

In some embodiments, the user device may be associated with a pharmacy management system.

In some embodiments, the user device may be associated with a wholesaler of a medicine corresponding to the medicine search code.

In some embodiments, the receiving of the medicine search code may be based on receiving unique scan code corresponding to the medicine search code. In some embodiments, the unique scan code may include a QR code associated with the medicine search code.

In some embodiments, the method 300 may further include a step of receiving, using the communication device 502, a point of sale data associated with user device. Further, the updating of the inventory data may be based on the point of sale data.

In some embodiments, the point of sale data may include one or more of a pharmacy management software data, a drug wholesaler data, and a distributor data.

In some embodiments, the receiving of the point of sale data may be facilitated by one or more of a platform access request received from the user device and transmitting, using the communication device 502, a data access request to the user device.

In some embodiments, the method 300 may further include a step of receiving, using the communication device 502, a power of attorney data associated with assigning power of attorney to the user. Further, the receiving of the medicine search code and the transaction type data may be based on the power of attorney data.

In some embodiments, the method 300 may further include a step of receiving, using the communication device 502, a request from the user device. Further, in some embodiments, the method 300 may include a step of transmitting, using the communication device 502, the power of attorney data to the user device based on the request.

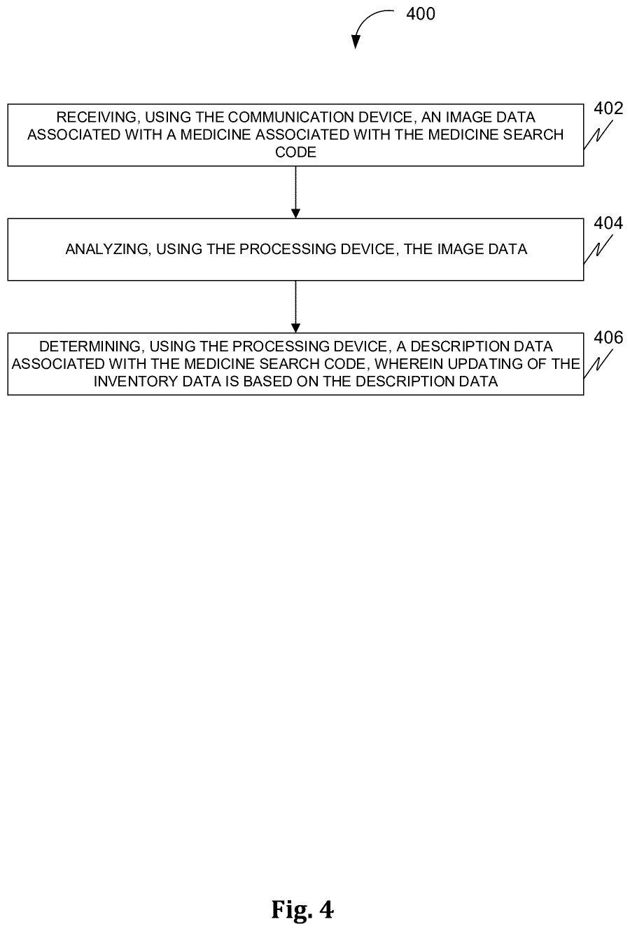

FIG. 4 illustrates a flowchart of a method 400 of facilitating pharmacy management including determining, using the processing device 504, a description data associated with the medicine search code, in accordance with some embodiments.

Further, in some embodiments, the method 400 further may include a step 402 of receiving, using the communication device 502, an image data associated with a medicine associated with the medicine search code. Further, in some embodiments, the method 400 further may include a step 404 of analyzing, using the processing device 504, the image data. Further, in some embodiments, the method 400 further may include a step 406 of determining, using the processing device 504, a description data associated with the medicine search code. Further, updating of the inventory data may be based on the description data.

In some embodiments, the analyzing of the image data may be based on a machine learning model.

In some embodiments, the machine learning model includes one or more of a supervised machine learning model, unsupervised machine learning model, a reinforcement learning model, and a semi supervised learning model.

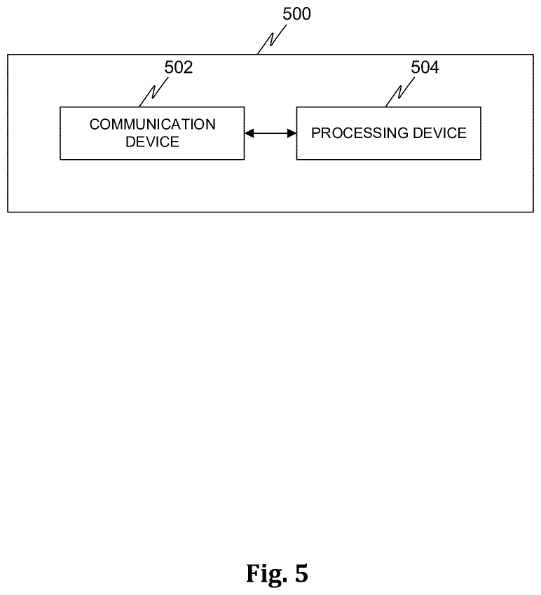

FIG. 5 illustrates a block diagram of a system 500 of facilitating pharmacy management, in accordance with some embodiments.

Accordingly, the system 500 may include a communication device 502. Further, the communication device 502 may be configured to receive two or more medicine data from one or more of a medicine database and a platform device. Further, each of the two or more medicine data corresponds to a unique medicine code. Further, the communication device 502 may be configured to receive a medicine search code and a transaction type data from a user device associated with a user. Further, the system 500 may include a processing device 504 communicatively coupled with the communication device 502. Further, the processing device 504 may be configured to compare the medicine search code and the unique medicine code. Further, the processing device 504 may be configured to identify an identified medicine data from the two or more medicine data based on the comparing. Further, the processing device 504 may be configured to update, an inventory data stored in a storage device based on the identified medicine data and the transaction type data.

In some embodiments, the communication device 502 may be further configured to receive the inventory data from the user device.

In some embodiments, the receiving of the transaction type data may be based on an external sensor data generated by an external sensor associated with a medicine corresponding to the medicine search code.

In some embodiments, the communication device 502 may be further configured to receive a description data associated with the medicine search code. Further, the updating of the inventory data may be based on the description data.

Further, in some embodiments, the communication device 502 may be configured to receive an image data associated with a medicine associated with the medicine search code. Further, the processing device 504 may be further configured to analyze the image data. Further, the processing device 504 may be further configured to determine a description data associated with the medicine search code. Further, updating of the inventory data may be based on the description data.

In some embodiments, the transaction type data includes indication of a receiving of a medicine corresponding to the medicine search code. Further, the updating of the inventory data may be based on the receiving of the medicine.

In some embodiments, the transaction type data includes indication of a dispensing of a medicine corresponding to the medicine search code. Further, the updating of the inventory data may be based on the dispensing of the medicine.

In some embodiments, the transaction type data includes indication of one or more of a loss and a damage of a medicine corresponding to the medicine search code.

Further, the updating of the inventory data may be based on a quantity associated with one or more of the loss and the damage of the medicine.

In some embodiments, the transaction type data includes indication of an expiration of a medicine corresponding to the medicine search code. Further, the updating of the inventory data may be based on the expiration of the medicine.

In some embodiments, the transaction type data includes indication of one or more of an audit and an amending of the inventory data.

In some embodiments, the transaction type data includes indication of a return of a medicine corresponding to the medicine search code. Further, the updating of the inventory data may be based on the return of the medicine.

In some embodiments, the storage device may be associated with a blockchain network.

In some embodiments, the processing device 504 may be further configured to generate a digital report data based on the inventory data.

Although the invention has been explained in relation to its preferred embodiment, it is to be understood that many other possible modifications and variations can be made without departing from the spirit and scope of the invention as hereinafter claimed.

What is claimed is:

1. A method of facilitating pharmacy management, the method comprising:
receiving, using a communication device, a plurality of medicine data from at least one of a medicine database and a platform device, wherein each of the plurality of medicine data corresponds to a unique medicine code;
receiving, using the communication device, a medicine search code, and a transaction type data from a user device associated with a user;
comparing, using a processing device, the medicine search code, and the unique medicine code;
identifying, using the processing device, an identified medicine data from the plurality of medicine data based on the comparing; and
updating, using the processing device, an inventory data stored in a storage device based on the identified medicine data and the transaction type data.

2. The method of claim 1 further comprising receiving, using the communication device, the inventory data from the user device.

3. The method of claim 1, wherein the receiving of the transaction type data is based on an external sensor data generated by an external sensor associated with a medicine corresponding to the medicine search code.

4. The method of claim 1 further comprises receiving, using the communication device, a description data associated with the medicine search code, wherein the updating of the inventory data is based on the description data.

5. The method of claim 1 further comprising:
receiving, using the communication device, an image data associated with a medicine associated with the medicine search code;
analyzing, using the processing device, the image data; and
determining, using the processing device, a description data associated with the medicine search code, wherein updating of the inventory data is based on the description data.

6. The method of claim 1, wherein the transaction type data comprises an indication of a receiving of a medicine corresponding to the medicine search code, wherein the updating of the inventory data is based on the receiving of the medicine.

7. The method of claim 1, wherein the transaction type data comprises an indication of a dispensing of a medicine corresponding to the medicine search code, wherein the updating of the inventory data is based on the dispensing of the medicine.

8. The method of claim 1, wherein the transaction type data comprises an indication of one or more of a loss and a damage of a medicine corresponding to the medicine search code, wherein the updating of the inventory data is based on a quantity associated with one or more of the loss and the damage of the medicine.

9. The method of claim 1, wherein the transaction type data comprises an indication of an expiration of a medicine corresponding to the medicine search code, wherein the updating of the inventory data is based on the expiration of the medicine.

10. The method of claim 1, wherein the storage device is associated with a blockchain network.

11. The system for facilitating pharmacy management, the system comprising:
a communication device configured to:
receive a plurality of medicine data from at least one of a medicine database and a platform device, wherein each of the plurality of medicine data corresponds to a unique medicine code;
receive a medicine search code and a transaction type data from a user device associated with a user;
a processing device communicatively coupled with the communication device, wherein the processing device is configured to:
compare the medicine search code and the unique medicine code;
identify an identified medicine data from the plurality of medicine data based on the comparing; and
update, an inventory data stored in a storage device based on the identified medicine data and the transaction type data.

12. The system of claim 11 wherein the communication device is further configured to receive the inventory data from the user device.

13. The system of claim 11, wherein the receiving of the transaction type data is based on an external sensor data generated by an external sensor associated with a medicine corresponding to the medicine search code.

14. The system of claim 11, wherein the communication device is further configured to receive a description data associated with the medicine search code, wherein the updating of the inventory data is based on the description data.

15. The system of claim 11, wherein communication device is configured to receive an image data associated with a medicine associated with the medicine search code, wherein the processing device is further configured to:
analyze the image data; and
determine a description data associated with the medicine search code, wherein updating of the inventory data is based on the description data.

16. The system of claim 11, wherein the transaction type data comprises an indication of a receiving of a medicine corresponding to the medicine search code, wherein the updating of the inventory data is based on the receiving of the medicine.

17. The system of claim 11, wherein the transaction type data comprises an indication of a dispensing of a medicine corresponding to the medicine search code, wherein the updating of the inventory data is based on the dispensing of the medicine.

18. The system of claim 11, wherein the transaction type data comprises an indication of one or more of a loss and a damage of a medicine corresponding to the medicine search code, wherein the updating of the inventory data is based on a quantity associated with one or more of the loss and the damage of the medicine.

19. The system of claim 11, wherein the transaction type data comprises an indication of an expiration of a medicine corresponding to the medicine search code, wherein the updating of the inventory data is based on the expiration of the medicine.

20. The system of claim 11, wherein the storage device is associated with a blockchain network.
